(19) United States
(12) Patent Application Publication
(10) Pub. No.: US 2025/0376712 A1
(43) Pub. Date: Dec. 11, 2025

(54) PAPER-BASED ANALYTICAL DEVICE FOR POTENTIOMETRIC ENZYME DETECTION

(71) Applicants: Hisham S.M. Abd-Rabboh, Abha (SA); Ayman H. Kamel, Islamabad (PK); Nasser S. Awwad, Abha (SA); Mohamed S. Hamdy, Abha (SA); Majed Bajaber, Abha (SA); Hala A. Ibrahim, Abha (SA)

(72) Inventors: Hisham S.M. Abd-Rabboh, Abha (SA); Ayman H. Kamel, Islamabad (PK); Nasser S. Awwad, Abha (SA); Mohamed S. Hamdy, Abha (SA); Majed Bajaber, Abha (SA); Hala A. Ibrahim, Abha (SA)

(21) Appl. No.: 18/734,058

(22) Filed: Jun. 5, 2024

(51) Int. Cl. C12Q 1/44; B01L 3/00; C12Q 1/00

(52) U.S. Cl. CPC C12Q 1/44; B01L 3/5023; C12Q 1/002; C12Q 1/005; C12Y 301/06008; B01L 2300/0645; B01L 2300/069; B01L 2300/0825

(57) ABSTRACT

A paper-based analytical device for potentiometric assay of Arylsulfatase A (ARSA) activity levels. The analytical device comprises a filter paper substrate supporting a potentiometric cell, a reference electrode, and an ion-selective electrode. The electrodes are constructed using an ink comprising carbon nanotubes and reduced graphene oxide (r-GO). The analytical device also includes a solid-contact potentiometric sensor for ARSA integrated with the electrodes and a polymeric membrane associated with the ion-selective electrode. The analytical device is capable of providing results from actual serum and whole blood samples. A method for monitoring blood ARSA levels and a method for fabricating the analytical device are also disclosed.

### COPYRIGHT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### 1) Field of the Invention

The present disclosure generally relates to the development and utilization of paper-based analytical devices, and more specifically, to the design and implementation of such analytical devices for the potentiometric assay of Arylsulfatase A enzyme activity.

### 2) Description of Related Art

The field of enzyme detection and quantification is of great interest in various sectors, including clinical diagnostics, environmental monitoring, and biotechnology research. Enzymes such as the lysosomal arylsulfatase enzymes, and particularly Arylsulfatase A (ARSA), are often used as biochemical indicators for various diseases, including several types of cancer.

Traditional methods for measuring enzyme activity, such as spectrophotometric and spectrofluorometric assays, involve the incubation of the enzyme with synthetic sulfated phenolic substrates, leading to the production of phenoxide ions. However, these methods have several limitations, including long incubation times, the inability to use buffered solutions, and the unsuitability for continuous monitoring of enzyme activity.

The development of potentiometric sensors has introduced a new paradigm in the measurement of ARSA activity. These sensors offer a non-destructive and passive analytical approach, converting ionic activity into electrical potential without the use of additional reagents or stimuli. They are known for their rapid response, reliability, simplicity, and cost-effectiveness, making them particularly attractive for downsizing and use in portable applications. Furthermore, the integration of solid-contact ion-selective electrodes (SC-ISEs) with nanomaterials has further enhanced the performance of these sensors, enabling more sensitive and stable measurements.

Molecularly Imprinted Polymers (MIPs) are another technology that has gained attention in the field of chemical sensing and biomedical diagnostics. MIPs are synthesized through a templating process that leaves behind cavities in the polymer matrix that are complementary in shape and functional groups to the target molecule. This lock-and-key mechanism confers high selectivity to MIPs, making them suitable for use in sensors that require high specificity. The robustness of MIPs, along with their resistance to harsh environmental conditions and their ability to operate over a wide pH range, has led to their widespread application in various fields.

Despite these advancements, the development of a portable, cost-effective, and disposable device for the rapid and precise measurement of enzyme activity remains a challenge. Such a device would be particularly useful in non-laboratory settings, where access to sophisticated laboratory equipment and trained personnel may be limited.

### BRIEF SUMMARY OF THE INVENTION

The present disclosure introduces a paper-based analytical device that uses potentiometric sensors and MIPs for the rapid and precise measurement of ARSA activity, offering a portable, cost-effective, and disposable solution for measuring ARSA activity levels even at trace levels. The analytical device utilizes a paper-based substrate to support a potentiometric cell, which includes a reference electrode and an ion-selective electrode, both constructed using an innovative ink composed of carbon nanotubes and reduced graphene oxide (r-GO). This configuration facilitates a solid-contact potentiometric sensor for ARSA, integrated with the electrodes, and a polymeric membrane associated with the ion-selective electrode.

The analytical device is designed to operate at a controlled temperature and pH, ensuring precise measurements of ARSA activity. It demonstrates a quick and consistent response over a linear range of 4-nitrocatechol sulfate (4-NCS) concentrations. The device provides results from actual serum and whole blood samples that are equivalent to those obtained from conventional standard methods makes it an ideal tool for direct, rapid, and affordable monitoring of ARSA levels in various settings, including non-laboratory environments. The paper-based design allows for easy disposal after a single use and is amenable to mass production using roll-to-roll printing processes, enhancing its accessibility and utility in clinical diagnostics and research. This innovative design allows for the direct, mediator-free, and pretreatment-free detection of ARSA, making it an ideal solution for use in non-laboratory settings.

The instant invention in one form is directed to a paper-based analytical device for potentiometric assay of Arylsulfatase A (ARSA) activity levels, comprising: a paper substrate supporting a potentiometric cell; a reference electrode and an ion-selective electrode formed on said substrate; electrodes constructed using an ink comprising carbon nanotubes and reduced graphene oxide (r-GO); a solid-contact potentiometric sensor for ARSA integrated with said electrodes; and a polymeric membrane associated with said ion-selective electrode.

In some aspects, the reference electrode is a paper Ag/AgCl electrode. In some aspects, the ion-selective electrode is responsive to 4-nitrocatechol sulfate (4-NCS) over a linear range of 1.0×10⁻² to 1.0×10⁻⁶ M. In some aspects, the ion-selective electrode exhibits an anionic slope of 59.5±0.3 mV/decade over a pH range of 3-6. In some aspects, the analytical device is configured to operate at a temperature of 37° C. and a pH of 5.0 for monitoring ARSA activity.

In some aspects, the analytical device demonstrates a linear relationship between the initial rate of substrate hydrolysis and ARSA activity within the range of 0.01 to 5.5 IU/L. In some aspects, the analytical device is capable of providing results from actual serum and whole blood samples that are equivalent to those obtained from conventional standard methods. In some aspects, the analytical device is disposable after a single use. In some aspects, the analytical device is fabricated using a roll-to-roll printing process suitable for mass production. In some embodiments, the paper substrate is a filter paper.

In some embodiments, the instant invention is directed to a method for monitoring blood ARSA levels comprising: applying a sample containing ARSA to the ion-selective electrode; measuring the potential difference between the reference electrode and the ion-selective electrode; and correlating the measured potential difference to the ARSA activity level in the sample. In some aspects, the sample is blood serum or whole blood. In some aspects, the potential difference is measured without the use of mediators or pretreatment of the sample.

In some embodiments, the instant invention is directed to a method for fabricating a paper-based analytical device for potentiometric assay of ARSA, comprising: patterning a filter paper substrate with wax to define zones for a reference electrode, an ion-selective electrode, and a sample application area; printing electrodes on the substrate using an ink comprising carbon nanotubes and r-GO; applying a polymeric membrane to the ion-selective electrode; and integrating a solid-contact potentiometric sensor for ARSA with the electrodes. In some aspects, the polymeric membrane includes a molecularly-imprinted polymer (MIP) specific for 4-NCS. In some aspects, the electrodes are printed using a stencil created by a laser cutter.

According to an aspect of the present disclosure, the device includes a paper-based analytical device for potentiometric assay of Arylsulfatase A (ARSA) activity levels. The device includes a filter paper substrate supporting a potentiometric cell, a reference electrode and an ion-selective electrode formed on the substrate. The electrodes are constructed using an ink including carbon nanotubes and reduced graphene oxide (r-GO). The device also includes a solid-contact potentiometric sensor for ARSA integrated with the electrodes, and a polymeric membrane associated with the ion-selective electrode.

According to other aspects of the present disclosure, the analytical device may include one or more of the following features. The reference electrode may be a paper Ag/AgCl electrode. The ion-selective electrode may be responsive to 4-nitrocatechol sulfate (4-NCS) over a linear range of 1.0×10−2 to 1.0×10−6 M. The ion-selective electrode may exhibit an anionic slope of 59.5±0.3 mV/decade over a pH range of 3-6. The analytical device may be configured to operate at a temperature of 37° C. and a pH of 5.0 for monitoring ARSA activity. The analytical device may demonstrate a linear relationship between the initial rate of substrate hydrolysis and ARSA activity within the range of 0.01 to 5.5 IU/L. The analytical device may be capable of providing results from actual serum and whole blood samples that are equivalent to those obtained from conventional standard methods. The analytical device may be disposable after a single use. The device may be fabricated using a roll-to-roll printing process suitable for mass production.

According to another aspect of the present disclosure, a method for monitoring blood ARSA levels using the analytical device includes applying a sample containing ARSA to the ion-selective electrode, measuring the potential difference between the reference electrode and the ion-selective electrode, and correlating the measured potential difference to the ARSA activity level in the sample.

According to other aspects of the present disclosure, the method may include one or more of the following features. The sample may be blood serum or whole blood. The potential difference may be measured without the use of mediators or pretreatment of the sample.

According to yet another aspect of the present disclosure, a method for fabricating a paper-based analytical device for potentiometric assay of ARSA includes patterning a filter paper substrate with wax to define zones for a reference electrode, an ion-selective electrode, and a sample application area, printing electrodes on the substrate using an ink including carbon nanotubes and r-GO, applying a polymeric membrane to the ion-selective electrode, and integrating a solid-contact potentiometric sensor for ARSA with the electrodes.

According to other aspects of the present disclosure, the method may include one or more of the following features. The polymeric membrane may include a molecularly-imprinted polymer (MIP) specific for 4-NCS. The electrodes may be printed using a stencil created by a laser cutter.

In summary, the present disclosure represents a leap forward in the field of enzyme activity measurement. By combining the specificity of MIPs with the convenience of paper-based potentiometric sensors, this technology promises to provide a quick, reliable, and cost-effective tool for monitoring ARSA activity levels, with potential applications in cancer diagnostics and other areas of clinical interest.

The device's portability, affordability, and disposability are particularly noteworthy. The use of paper as the substrate material not only reduces the cost and weight of the device but also allows for easy disposal after a single use, addressing environmental concerns associated with traditional glass-based sensors. Furthermore, the planar form of the device is amenable to mass production using roll-to-roll printing processes, which can facilitate widespread distribution and accessibility of this technology.

These and other objects, features, and advantages of the present invention will become more readily apparent from the attached drawings and the detailed description of the preferred embodiments, which follow.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 is a schematic of an analytical device for potentiometric detection of ARSA activity levels, according to an embodiment of this invention;

FIG. 2A is a schematic providing further details of an analytical device for potentiometric detection of ARSA activity levels, according to an embodiment of this invention;

FIG. 2B is a cross-section of the analytical device shown in FIG. 2A for potentiometric detection of ARSA activity levels;

FIG. 3 is a process flow diagram for a method for fabricating a paper-based analytical device for potentiometric assay of ARSA; and

FIG. 4 is a process flow diagram showing a method for monitoring blood ARSA levels using the analytical device described in this disclosure.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The lysosomal arylsulfatase enzymes (arylsulfatase sulfohydrolase, EC 3.1.6.1) are possible biochemical indicators for several cancer types. Numerous cancers, including those of the skin, the breast, the bladder, the womb, and the prostate, as well as lymphogranulomatosis (Hodgkin's disease), have been linked to an increase in the enzyme's activity. Arylsulfatase activity is often measured using spectrophotometric and spectrofluorometric rate measurements of the phenoxide ion generated when the enzyme is incubated with synthetic sulfated phenolic substrates.

One of the most effective substrates for spectrophotometric evaluation of arylsulfatase activity has been 4-nitrocatechol sulfate (CNCS). The requirement for a lengthy incubation reaction time (90-180 min), the restriction to unbuffered test solutions, and the inapplicability for continuous enzyme monitoring are all inherent drawbacks. Although nitro quinol sulfate substrate has proven to be suitable for continuous assay of ARSA, its sensitivity is 0.4 times lower than that of 4-NCS and it is unstable in alkaline conditions. Repeated scanning in the ultraviolet-visible range between two wavelengths or at a fixed wavelength using substrates made of 4-nitrophenyl sulfate has been proposed and tested. The approach can be used across a wide pH range; however, it is not sensitive enough to measure enzyme activity that is less than 30 units. There are drawbacks to all the published kinetic spectrophotometric and fluorometric techniques. They typically do not apply to test solutions that are turbid, colored, buffered, and samples that contain quenching foreign compounds. Potentiometric approaches can be used to get rid of all these limitations.

The present disclosure provides a detailed description of a paper-based analytical device designed for the potentiometric assay of Arylsulfatase A (ARSA) activity levels. This device is particularly advantageous for its portability, affordability, and disposability, making it an ideal solution for use in non-laboratory settings and for mass production.

In some aspects, the analytical device may include a filter paper substrate supporting a potentiometric cell, a reference electrode and an ion-selective electrode formed on the substrate, and electrodes constructed using an ink comprising carbon nanotubes and reduced graphene oxide (r-GO). The analytical device may also include a solid-contact potentiometric sensor for ARSA integrated with the electrodes, and a polymeric membrane associated with the ion-selective electrode.

In some cases, the analytical device may be configured to operate at a temperature of 37° C. and a pH of 5.0 for monitoring ARSA activity. The analytical device may demonstrate a linear relationship between the initial rate of substrate hydrolysis and ARSA activity within the range of 0.01 to 5.5 IU/L. In some aspects, the analytical device may be capable of providing results from actual serum and whole blood samples that are equivalent to those obtained from conventional standard methods. The analytical device may be disposable after a single use and may be fabricated using a roll-to-roll printing process suitable for mass production.

The present disclosure also relates to a method for monitoring blood ARSA levels using the analytical device. In some aspects, the method may include applying a sample containing ARSA to the ion-selective electrode, measuring the potential difference between the reference electrode and the ion-selective electrode, and correlating the measured potential difference to the ARSA activity level in the sample.

Further, the present disclosure relates to a method for fabricating a paper-based analytical device for potentiometric assay of ARSA. In some cases, the method may include patterning a paper substrate with wax to define zones for a reference electrode, an ion-selective electrode, and a sample application area, printing electrodes on the substrate using an ink comprising carbon nanotubes and r-GO, applying a polymeric membrane to the ion-selective electrode, and integrating a solid-contact potentiometric sensor for ARSA with the electrodes.

The device and methods of the present disclosure may provide several technical benefits. For instance, the analytical device may offer a cost-effective, portable, and easy-to-use solution for monitoring ARSA activity levels in various samples. The analytical device may also provide accurate and reliable results, which may be particularly beneficial in clinical settings for diagnosing and monitoring metabolic diseases. The methods for monitoring ARSA levels and fabricating the analytical device may be straightforward and efficient, potentially facilitating widespread adoption and use of the analytical device in various fields, including healthcare, environmental monitoring, and biotechnology research.

In some aspects, the paper-based analytical device may include a paper substrate that supports a potentiometric cell. The potentiometric cell may be formed on the substrate and may include a reference electrode and an ion-selective electrode. In some cases, the reference electrode may be a paper Ag/AgCl electrode. This type of electrode may provide a stable reference potential, which may be beneficial for accurate and reliable measurements of ARSA activity levels.

The ion-selective electrode, in some aspects, may be responsive to 4-nitrocatechol sulfate (4-NCS). The responsiveness of the ion-selective electrode to 4-NCS may be over a linear range of 1.0×10⁻² to 1.0×10⁻⁶ M. This range may allow for the detection and quantification of a wide range of ARSA activity levels in various samples, including but not limited to blood serum and whole blood samples.

In some cases, the ion-selective electrode may exhibit an anionic slope of 59.5±0.3 mV/decade over a pH range of 3-6. This operational characteristic may provide a consistent and predictable response to changes in 4-NCS concentration, which may be beneficial for accurate and reliable measurements of ARSA activity levels.

The electrodes of the potentiometric cell may be constructed using an ink comprising carbon nanotubes and reduced graphene oxide (r-GO). The use of carbon nanotubes and r-GO in the ink may provide several technical benefits. For instance, carbon nanotubes and r-GO may enhance the electrical conductivity of the electrodes, which may improve the sensitivity and accuracy of the analytical device. Additionally, carbon nanotubes and r-GO may provide a robust and durable structure for the electrodes, which may be beneficial for the longevity and reliability of the analytical device.

The electrodes of the potentiometric cell may be constructed using an ink that includes carbon nanotubes and reduced graphene oxide (r-GO). The carbon nanotubes and r-GO may contribute to the electrical conductivity of the electrodes, potentially enhancing the sensitivity and accuracy of the analytical device. Furthermore, the robust and durable structure provided by the carbon nanotubes and r-GO may contribute to the longevity and reliability of the analytical device. The analytical device may also include a solid-contact potentiometric sensor for ARSA integrated with the electrodes. This sensor may be designed to detect and measure ARSA activity levels, providing a direct and accurate assessment of these levels in various samples. The integration of the sensor with the electrodes may facilitate efficient and reliable measurements, potentially improving the overall performance of the analytical device.

In some aspects, the analytical device may be configured to operate at a temperature of 37° C. and a pH of 5.0 for monitoring ARSA activity. This operational configuration may be particularly suitable for detecting and measuring ARSA activity levels in biological samples, such as blood serum and whole blood. The ability of the analytical device to operate at these specific conditions may contribute to its accuracy and reliability, potentially providing results that are equivalent to those obtained from conventional standard methods. The analytical device may demonstrate a linear relationship between the initial rate of substrate hydrolysis and ARSA activity within the range of 0.01 to 5.5 IU/L. This functional characteristic may allow for the precise quantification of ARSA activity levels in various samples. The linear relationship may provide a straightforward and intuitive way to interpret the results, potentially facilitating the use of the analytical device in various settings, including clinical, environmental, and research contexts.

The analytical device may include a polymeric membrane associated with the ion-selective electrode. This polymeric membrane may play a pivotal role in the operation of the analytical device. Specifically, the polymeric membrane may serve as an interface between the ion-selective electrode and the sample containing ARSA. This interface may facilitate the selective interaction of the ion-selective electrode with 4-NCS, a compound that may be indicative of ARSA activity levels. By facilitating this selective interaction, the polymeric membrane may contribute to the accuracy and reliability of the analytical device in measuring ARSA activity levels.

In some cases, the polymeric membrane may include a molecularly-imprinted polymer (MIP) specific for 4-NCS. The MIP may be designed to have a high affinity for 4-NCS, potentially enhancing the selectivity of the ion-selective electrode for this compound. This high selectivity may improve the sensitivity of the analytical device, potentially allowing for the detection and quantification of a wide range of ARSA activity levels. Furthermore, the use of a MIP specific for 4-NCS may reduce the likelihood of interference from other compounds present in the sample, potentially improving the selectivity and overall performance of the analytical device. The polymeric membrane may be applied to the ion-selective electrode during the fabrication of the analytical device. This application may be performed using various techniques, such as spin coating, dip coating, spray coating, contact printing, contact transfer or direct lamination. The choice of technique may depend on various factors, including but not limited to the properties of the polymeric membrane, the characteristics of the ion-selective electrode, and the desired performance characteristics of the analytical device. Regardless of the technique used, the application of the polymeric membrane to the ion-selective electrode may be performed in a manner that ensures a uniform and stable interface, potentially contributing to the consistency, selectivity, and reliability of the analytical device measurements.

The paper-based analytical device may be configured to operate under specific conditions for monitoring ARSA activity. For instance, the analytical device may be designed to function at a temperature of 37° C. This temperature may be particularly suitable for biological samples, such as blood serum and whole blood, as it closely mimics the human body temperature. In addition to temperature, the analytical device may also be configured to operate at a specific pH. In some cases, the analytical device may function at a pH of 5.0. This pH level may be ideal for the detection and measurement of ARSA activity levels, as it may provide an environment that facilitates the interaction between the ion-selective electrode and 4-NCS, a compound indicative of ARSA activity levels.

The analytical device may be capable of providing results from actual serum and whole blood samples that are equivalent to those obtained from conventional standard methods. This capability may be particularly beneficial in clinical settings, where accurate and reliable results are paramount. The ability of the analytical device to provide equivalent results may be attributed to its design and operational characteristics, including but not limited to the use of carbon nanotubes and r-GO in the electrodes, the integration of a solid-contact potentiometric sensor for ARSA, and the application of a polymeric membrane to the ion-selective electrode. The analytical device may be disposable after a single use. This operational characteristic may offer several advantages. For instance, it may eliminate the risk of cross-contamination between samples, which may be particularly beneficial in clinical and environmental settings. Additionally, the disposability of the analytical device may simplify its use, as it may not require cleaning or maintenance after use. This feature may make the analytical device particularly suitable for use in field testing or in settings where access to cleaning facilities may be limited.

The paper-based analytical device may be capable of providing results from actual serum and whole blood samples. These samples may be applied directly to the ion-selective electrode of the analytical device without the use of mediators or pretreatment. This direct application may simplify the operation of the analytical device, potentially making it more user-friendly and accessible to a wide range of users, including those without specialized training or equipment.

The analytical device may measure the potential difference between the reference electrode and the ion-selective electrode in the presence of the sample. This measurement may be performed without the use of mediators or pretreatment of the sample, potentially simplifying the operation of the analytical device and reducing the risk of errors or inaccuracies due to sample manipulation.

In some cases, the analytical device may provide results that are equivalent to those obtained from conventional standard methods. This equivalence may be demonstrated across a range of ARSA activity levels and in various types of samples, including but not limited to blood serum and whole blood. The ability of the analytical device to provide equivalent results may be attributed to its design and operational characteristics, including the use of carbon nanotubes and r-GO in the electrodes, the integration of a solid-contact potentiometric sensor for ARSA, and the application of a polymeric membrane to the ion-selective electrode. This equivalence may provide confidence in the accuracy and reliability of the analytical device measurements, potentially facilitating its adoption and use in various settings, including clinical, environmental, and research contexts.

The analytical device may be fabricated using a roll-to-roll printing process suitable for mass production. This fabrication process may involve printing the electrodes on the filter paper substrate using an ink comprising carbon nanotubes and r-GO. The roll-to-roll printing process may allow for the efficient and cost-effective production of the analytical device on a large scale, potentially facilitating its widespread adoption and use. This process may also contribute to the consistency and reliability of the analytical device, as it may ensure a uniform and precise application of the ink on the substrate. The sample applied to the ion-selective electrode may contain ARSA. This enzyme may be present in various types of samples, including but not limited to blood serum and whole blood. The presence of ARSA in the sample may be indicative of its activity levels, which may be a measure of metabolic health or disease status. The ability of the analytical device to detect and measure ARSA activity levels in the sample may provide a direct and accurate assessment of these levels, potentially facilitating the diagnosis and monitoring of metabolic diseases.

The method for monitoring blood ARSA levels using the paper-based analytical device may involve the application of a sample containing ARSA to the ion-selective electrode. This application may be performed directly, without the use of mediators or pretreatment of the sample. The sample may contain ARSA and may be a biological sample, such as blood serum or whole blood. The direct application of the sample to the ion-selective electrode may facilitate the interaction between the electrode and 4-NCS, a compound indicative of ARSA activity levels. This interaction may result in a potential difference between the reference electrode and the ion-selective electrode, which may be measured by the analytical device.

The potential difference may be measured without the use of mediators or pretreatment of the sample. This measurement may be performed using the solid-contact potentiometric sensor for ARSA integrated with the electrodes. The sensor may detect and measure the potential difference, providing a direct and accurate assessment of ARSA activity levels in the sample. The measurement of the potential difference may be performed in a manner that ensures a consistent and reliable response to changes in 4-NCS concentration, potentially contributing to the accuracy and reliability of the device measurements.

The measured potential difference may be correlated to the ARSA activity level in the sample. This correlation may be based on the linear relationship between the initial rate of substrate hydrolysis and ARSA activity within the range of 0.01 to 5.5 IU/L. The correlation may provide a straightforward and intuitive way to interpret the results, potentially facilitating the use of the analytical device in various settings, including clinical, environmental, and research contexts.

In some cases, the electrodes of the analytical device may be printed using a stencil created by a laser cutter. This printing process may involve the application of an ink comprising carbon nanotubes and r-GO to the stencil, followed by the transfer of the ink to the filter paper substrate. The use of a laser-cut stencil may ensure a precise and uniform application of the ink, potentially enhancing the electrical conductivity of the electrodes and improving the overall performance of the analytical device. The printing process may be performed in a manner that ensures a robust and durable structure for the electrodes, potentially contributing to the longevity and reliability of the analytical device. The fabrication of the paper-based analytical device may involve several steps. One of these steps may include patterning a filter paper substrate with wax to define zones for a reference electrode, an ion-selective electrode, and a sample application area. The patterning of the substrate may be performed using various techniques, such as wax printing, wax dipping, or wax spraying. The choice of technique may depend on various factors, including but not limited to the properties of the filter paper substrate, the characteristics of the wax, and the desired performance characteristics of the analytical device. Regardless of the technique used, the patterning of the substrate may be performed in a manner that ensures a precise and uniform definition of the zones, potentially contributing to the consistency and reliability of the analytical device measurements.

The fabrication of the analytical device may also involve printing electrodes on the substrate using an ink comprising carbon nanotubes and r-GO. This printing process may be performed using various techniques, such as screen printing, inkjet printing, or flexographic printing. The choice of technique may depend on various factors, including but not limited to the properties of the ink, the characteristics of the substrate, and the desired performance characteristics of the analytical device. Regardless of the technique used, the printing of the electrodes may be performed in a manner that ensures a robust and durable structure for the electrodes, potentially contributing to the longevity and reliability of the analytical device.

In some cases, the fabrication of the analytical device may involve integrating a solid-contact potentiometric sensor for ARSA with the electrodes. This integration may be performed using various techniques, such as lamination, bonding, or welding. The choice of technique may depend on various factors, including but not limited to the properties of the sensor, the characteristics of the electrodes, and the desired performance characteristics of the analytical device. Regardless of the technique used, the integration of the sensor with the electrodes may be performed in a manner that ensures a robust and durable structure for the sensor, potentially contributing to the longevity and reliability of the analytical device.

The following paragraphs provide a comprehensive understanding of the various aspects of the analytical device, its fabrication, and its operation.

Synthesis of Molecularly Imprinted Polymers (MIPs): The core component of the ion-selective electrode is the Molecularly Imprinted Polymer (MIP), which is synthesized using a free-radical polymerization process. The MIPs are created by polymerizing functional monomers in the presence of a template molecule, which is later removed to leave behind cavities that are complementary in shape and chemical functionality to the target molecule, in this case, ARSA. This process ensures that the MIPs have a high affinity for ARSA, providing the specificity and selectivity that is central to the analytical device's function.

In one exemplary embodiment, MIPs are produced by the following process of free radical polymerization: 3.0 mmol of methacrylic acid (MAA) monomer and 1.0 mmol of 4-NCS were diluted in 20 mL of acetonitrile. 3.0 mmol of ethyleneglycol dimethacrylate (EGDMA) and 80 mg of benzoyl benzoate (PBO) were mixed and added to the reaction mixture. A nitrogen gas stream was diffused into the cocktail solution for 10 minutes before being carefully sealed to remove the dissolved oxygen. The tube spent 18 hours in a water bath set at 70° C. for extensive polymerization. The template was eliminated using batch mode solvent extraction with ethanol/acetic acid (8:2, v/v) and ethanol after the polymer had generated. The resulting polymer spent the entire night drying at 40° C. Non-imprinted polymer (NIP) particles were produced by repeating the prior methods in the absence of the template molecule.

Fabrication of the Paper-Based Analytical Device: The device is constructed on a paper substrate, which serves as a cost-effective and lightweight platform for the potentiometric cell. The paper is patterned with hydrophobic wax to create distinct zones for the reference and ion-selective electrodes, as well as microfluidic channels that facilitate the movement of the sample. This patterning is achieved through wax printing, which is a simple and scalable process suitable for mass production.

In one exemplary embodiment, fabrication of paper based analytical device was done using following method: All paper-based zones and microfluidic channels were patterned using wax printing on chromatographic paper (Whatman 1-Chr). The width of each printed wax line is 2 mm. The paper was first heated to 80° C. in the oven to melt the wax, which was then allowed to seep into every crevice of the paper. The Ag/AgCl ink stencils used to create the electrodes were printed on wax-printed paper. Using a laser cutter, the printing stencil was created and sliced into a frisket film (Grafix, low tack). The paper was covered with the stencil, and all empty places were subsequently filled with ink. After applying the ink, the paper was baked at 100° C. for 10 minutes to help the ink cling to the paper's surface. To increase the hydrophilicity of each electrode zone, 1 μL of 3-aminopropyldimethylethoxysilane (APDES, 2 wt %) was added to each zone.

Electrode Construction: The electrodes are printed onto the paper substrate using an ink that contains a mixture of carbon nanotubes and reduced graphene oxide (r-GO). This ink formulation provides a conductive path for electron transfer and a stable environment for the polymeric membrane of the ion-selective electrode. The reference electrode is typically a paper-based Ag/AgCl electrode, which offers a stable and reliable reference potential for the measurements.

Membrane Preparation: The sensing membrane is a composite of the MIP, a lipophilic additive, polyvinyl chloride (PVC), a plasticizer, and r-GO, which acts as a solid-contact transducer. The membrane is carefully cast onto the transducer layer and allowed to dry, forming a robust and selective layer that is responsive to the presence of ARSA in the sample.

The MIP receptor, the lipophilic additive ETH500, PVC, o-NPOE, and the solid-contact transducer reduced graphene oxide (rGO) are frequently found in the sensing membrane. In one exemplary embodiment, the membrane preparation was done by following process: The sensing membrane was dissolved in 2 mL of THF and included 1.5 wt % of the MIP, 0.5 wt % of ETH, 10 μL of rGO, 33.0 wt % of PVC, and 65.0 wt % of o-NPOE. The membrane mixture was drop-cast in 100 μL onto the transducer layer, where it was left to dry for 2 h. The same procedures were followed again without adding the solid-contact material (rGO) to create the un-modified electrodes.

Electrode Fabrication and Potential Measurements: The paper-based device includes three main zones: the reference zone, the central zone, and the sampling zone. The potential difference between the reference electrode and the ion-selective electrode is measured using a standard mV/pH meter. This potential difference is indicative of the ARSA activity levels in the sample, which can be correlated to the concentration of ARSA present.

In one exemplary embodiment, the following process was used for electrode fabrication and potential measurements: Three distinct zones are included in the developed paper gadget. They are divided into reference, central, and sampling zones. On the sample, center, and reference zones, respectively, the inner reference, sample, and external reference solutions were spotted. At 25° C., all measurements were made. The potential difference between the two paper electrodes for aqueous 4-NCS samples with various 4-NCS ion concentrations was measured using an mV/pH meter. Aqueous 1 M KCl served as the reference solution for the external paper-based reference electrode. The solutions were applied to each zone in a total volume of 10 μL. In around 20 seconds, the solutions spread into the main mixing area.

Potentiometric Determination of Arylsulfatase: The potentiometric determination of ARSA is performed by monitoring the rate of potential change upon the addition of the enzyme to a solution containing the substrate 4-NCS. The device is designed to operate at a controlled temperature and pH, which are optimized for the activity of ARSA. The rate of potential change is directly proportional to the enzyme activity, allowing for the quantification of ARSA levels in the sample.

In one exemplary embodiment, the following method was used for potentiometric determination of Arylsulfatase: In a 50 mL double jacketed reaction cell that was thermostated at 37±0.1° C., 0.1 mL of 1×10⁻³ M 4-NCS and 4.9 mL of 0.1 M sodium acetate-acetic acid buffer of pH 5.0 were combined. For the analysis, aliquots of this solution were added to the electrochemical paper-based device. Following the acquisition of a steady potential reading, 10 μL aliquots containing 0.1-6.0 IU of the enzyme arylsulfatase was introduced. The maximum initial rates of potential change, given in mV/min, were graphically computed using the rate part of the rate curves, which were shown. The calibration graph was applied to later assessments of an unidentified enzyme's activity. Under analogous circumstances, a control experiment was run without the enzyme.

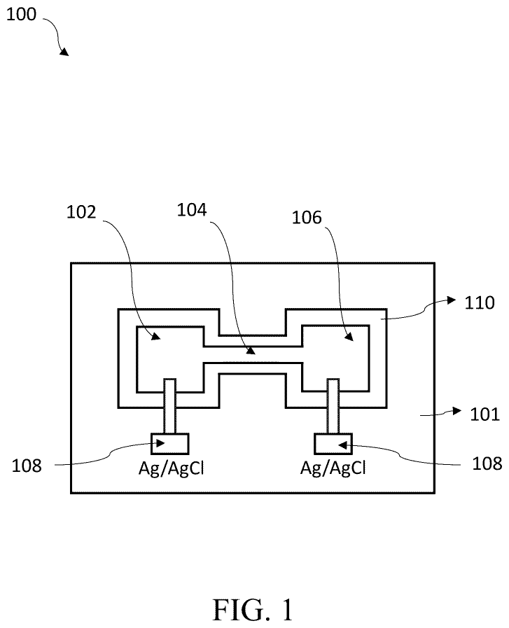

FIG. 1 shows a schematic of an analytical device 100 for potentiometric detection of ARSA activity levels. Various components of the analytical device have been described in detail in previous sections. The analytical device is a potentiometric cell constructed on a paper substrate 101 and comprises a sample zone 102 connected to a reference zone 106 via a central zone 104. Each of the sample zone and the reference zone have a Ag/AgCl reference electrode 108. The boundaries of the analytical device are defined by a wax layer patterned on the paper substrate. The paper substrate soaks up the solvent from the sample and thus provides a medium for ionic transport. The central zone 104 acts as an ion channel between the sample zone 102 and reference zone 106.

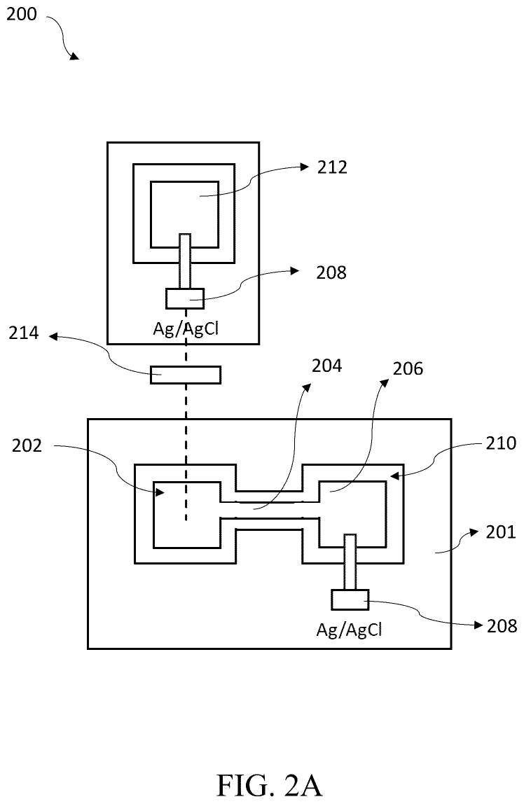

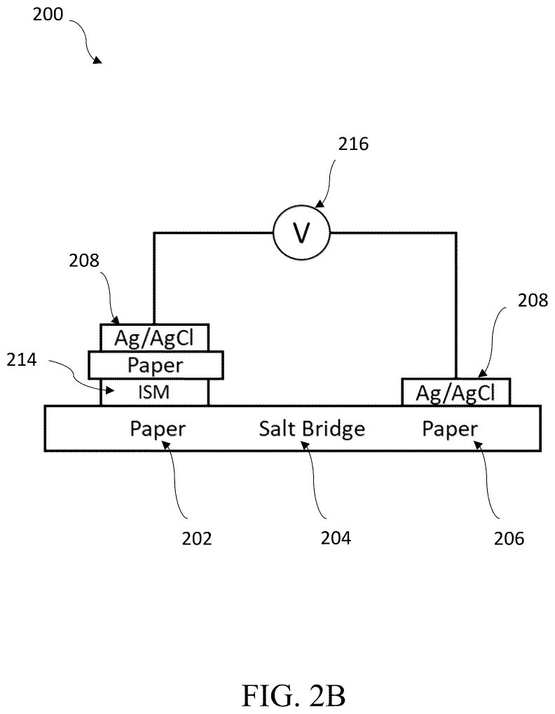

FIG. 2A and 2B provide further details of an analytical device 200 as described in various embodiments of this invention. FIG. 2A is a schematic showing detailed view of an analytical device for potentiometric detection of ARSA activity levels on a paper substrate 201. FIG. 2B is a cross-section of the analytical device shown in FIG. 2A for potentiometric detection of ARSA activity levels.

The device 200 comprises a reference zone 206 comprising KCl and having an Ag/AgCl reference electrode 208. The ion channel 204 is situated in the central zone which connects the reference electrode to the sample zone 202. The sample zone 202 further comprises an inner reference zone 212 including an Ag/AgCl electrode, and an ion selective electrode underneath which includes printed carbon nanotube/r-GO electrodes and an ion-selective polymeric membrane 214 laminated on the printed electrodes. The ion selective membrane can be fabricated using a MIP as described in various embodiments of the invention.

As shown in the cross-section depicted in FIG. 2B, the ion channel 204 acts as a salt bridge. The boundaries of the device are defined by a wax layer 210 patterned on the paper substrate. The paper substrate soaks up the solvent from the sample and thus provides a medium for ionic transport. 216 is the potential difference measured by the potentiometer between the sample and the reference when the sample analyte is filled in the sample zone. The rate of potential change is directly proportional to the enzyme activity, allowing for the quantification of ARSA levels in the sample.

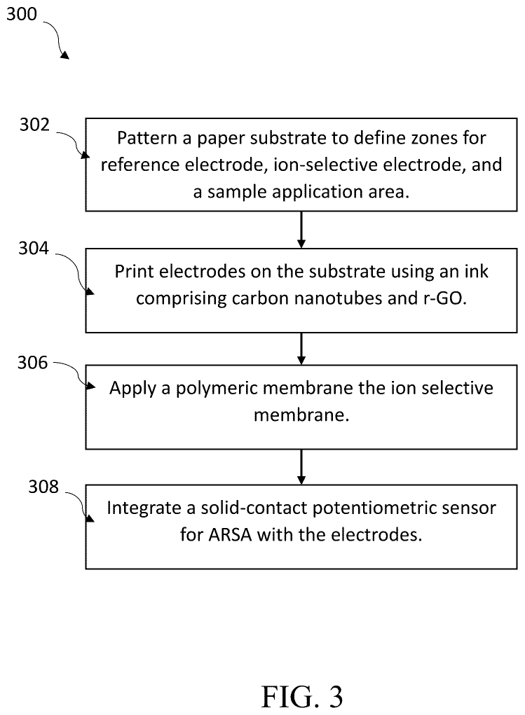

FIG. 3 shows a process flow diagram for a method for fabricating a paper-based analytical device for potentiometric assay of ARSA. In the step 302 a paper substrate is patterned with wax to define zones for a reference electrode, an ion-selective electrode, and a sample application area as described in previous sections. In step 304, electrodes on the substrate are printed using an ink comprising carbon nanotubes and r-GO as described in detail in previous sections of the disclosure. In step 306, a polymeric membrane is applied to the ion-selective electrode as described in previous sections of this disclosure. In step 308, a solid-contact potentiometric sensor for ARSA is integrated with the electrodes as described in previous sections.

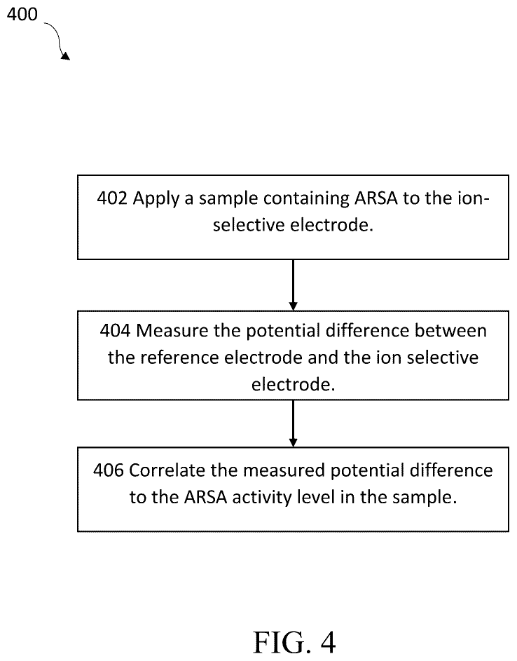

FIG. 4 shows a process flow diagram showing A method for monitoring blood ARSA levels using the analytical device described in this disclosure. In step 402 a sample containing ARSA is applied to the ion-selective electrode as described previously in this disclosure. In step 404 the potential difference is measured between the reference electrode and the ion-selective electrode as shown in FIG. 2B and described previously. In step 406 the measured potential difference is correlated to the ARSA activity level in the sample.

Alternative Embodiments: The following paragraphs describe alternative embodiments than those described in previous sections.

Variations in Material Composition: The electrodes in the device are constructed using an ink that contains a mixture of carbon nanotubes and reduced graphene oxide (r-GO). However, other conductive materials could be used in place of or in addition to these components. For example, other types of nanotubes or graphene derivatives could be used. Alternatively, other conductive materials such as silver nanoparticles, gold nanoparticles, or conductive polymers could be used. These variations could potentially improve the conductivity, stability, or other properties of the electrodes.

Variations in Fabrication Methods: The device is currently fabricated using wax printing to pattern the paper substrate and create the electrodes. However, other fabrication methods could be used. For example, screen printing, inkjet printing, or 3D printing could be used to create the electrodes. These alternative fabrication methods could potentially offer advantages in terms of precision, scalability, or cost.

Variations in Membrane Composition: The sensing membrane is currently a composite of the MIP, a lipophilic additive, PVC, a plasticizer, and r-GO. However, other materials could be used in the membrane. For example, other types of polymers could be used in place of PVC. Alternatively, other types of additives or plasticizers could be used. These variations could potentially improve the selectivity, sensitivity, or other properties of the membrane.

Variations in Device Configuration: The device currently includes three main zones: the reference zone, the central zone, and the sampling zone. However, the configuration of these zones could be varied. For example, additional zones could be added to the device, such as a control zone or a calibration zone. Alternatively, the size, shape, or arrangement of the zones could be varied. These variations could potentially improve the usability, accuracy, or other aspects of the device.

Variations in Operational Parameters: The device is currently designed to operate at a controlled temperature and pH. However, the operational parameters could be varied. For example, the device could be designed to operate at different temperatures or pH levels. Alternatively, other operational parameters could be controlled, such as the humidity or the pressure. These variations could potentially expand the range of conditions under which the device can be used.

Variations in Target Molecules: While the current embodiment of the device is optimized for the detection of ARSA, the device could potentially be adapted for the measurement of other enzymes or biomolecules. By changing the MIP and adjusting the membrane composition, the device could be tailored to detect a wide range of targets. This could potentially expand the utility of the device in clinical diagnostics and research.

Advantages of the Paper-Based Analytical Device: The paper-based analytical device offers several advantages over traditional methods for measuring ARSA activity. Its rapid response time and the ability to perform measurements without the use of additional reagents or pretreatment steps make it a convenient tool for point-of-care diagnostics. Additionally, the device's disposability ensures that there is no risk of cross-contamination between samples.

Environmental Considerations: The use of paper as the substrate material not only reduces the cost and environmental impact of the device but also simplifies the disposal process. After a single use, the device can be discarded without the concerns associated with the disposal of traditional glass-based sensors.

Mass Production and Accessibility: The planar form of the device is amenable to roll-to-roll printing processes, which can facilitate the mass production of the sensors. This production method can lead to widespread distribution and accessibility of the technology, particularly in resource-limited settings where traditional laboratory equipment is not available.

Clinical Implications: The ability to measure ARSA activity levels quickly and accurately has implications for cancer diagnostics, as ARSA has been identified as a potential biochemical marker for various types of cancer. The paper-based analytical device could therefore play a role in the early detection and monitoring of cancer, improving patient outcomes.

Potential for Further Development: While the current embodiment of the device is optimized for the detection of ARSA, the platform could potentially be adapted for the measurement of other enzymes or biomolecules. By changing the MIP and adjusting the membrane composition, the device could be tailored to detect a wide range of targets, expanding its utility in clinical diagnostics and research.

Alternative Applications: The following paragraphs provide details on other applications of the device described herein: environmental monitoring, food and beverage industry, pharmaceutical industry, biotechnology research, and agriculture.

Environmental Monitoring: The paper-based analytical device could be used in environmental monitoring, specifically in the detection of enzyme activity in soil or water samples. The portability and disposability of the device make it ideal for field testing, and its sensitivity to a range of enzyme concentrations could provide valuable data on the health of ecosystems. For instance, the device could be used to monitor the activity of ARSA or other enzymes in soil samples as an indicator of soil health and fertility.

Food and Beverage Industry: The device could be applied in the food and beverage industry for quality control and safety testing. Enzyme activity is often a measure of freshness or spoilage in food products, and this device could provide a quick, affordable, and easy-to-use method for such testing. For example, it could be used to monitor enzyme activity in milk to detect spoilage, or in fruit juices to ensure they have been properly pasteurized.

Pharmaceutical Industry: In the pharmaceutical industry, the device could be used in drug development and testing. Many drugs work by interacting with enzymes in the body, and this device could provide a way to measure these interactions. For example, it could be used to monitor the activity of ARSA or other enzymes in response to a new drug, helping researchers to understand the drug's mechanism of action and potential side effects.

Biotechnology Research: The device could be used in biotechnology research, where enzyme activity is often a focus of study. The device's sensitivity and specificity could make it a valuable tool for researchers studying the role of enzymes in biological processes or developing new biotechnological applications. For example, it could be used in the study of genetic diseases that involve enzyme deficiencies, or in the development of biofuels that rely on enzyme activity.

Agriculture: In agriculture, the device could be used to monitor soil health and fertility, as enzyme activity is often a measure of these factors. The device's portability and disposability make it ideal for field testing, and its sensitivity to a range of enzyme concentrations could provide valuable data for farmers and agronomists. For example, it could be used to monitor the activity of ARSA or other enzymes in soil samples as an indicator of nutrient availability or microbial activity.

The paper-based analytical device described herein represents a leap forward in the field of enzyme activity measurement. By combining the specificity of MIPs with the convenience of paper-based potentiometric sensors, this technology promises to provide a quick, reliable, and cost-effective tool for monitoring ARSA activity levels, with potential applications in cancer diagnostics and other areas of clinical interest.

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

A number of implementations have been described. Nevertheless, it will be understood that various modifications may be made without departing from the spirit and scope of the disclosure. Accordingly, other implementations are within the scope of the following claims.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An analytical device for potentiometric assay of ARSA activity levels, the analytical device comprising:
a paper substrate supporting a potentiometric cell;
a reference electrode and an ion-selective electrode formed on the paper substrate,
wherein the reference electrode and the ion-selective electrode are constructed using an ink comprising carbon nanotubes and reduced graphene r-GO;
a solid-contact potentiometric sensor for ARSA integrated with the reference electrode and the ion-selective electrode; and
a polymeric membrane associated with said ion-selective electrode.

2. The analytical device of claim 1, wherein the reference electrode is a paper Ag/AgCl electrode.

3. The analytical device of claim 1, wherein the ion-selective electrode is responsive to 4-nitrocatechol sulfate (4-NCS) over a linear range of 1.0×10⁻² to 1.0×10⁻⁶ M.

4. The analytical device of claim 1, wherein the ion-selective electrode exhibits an anionic slope of 59.5±0.3 mV/decade over a pH range of 3-6.

5. The analytical device of claim 1, wherein the polymeric membrane includes a molecularly-imprinted polymer (MIP) specific for 4-NCS.

6. The analytical device of claim 1, wherein the analytical device is configured to operate at a temperature of 37° C. and a pH of 5.0 for monitoring ARSA activity.

7. The analytical device of claim 1, wherein the analytical device demonstrates a linear relationship between an initial rate of substrate hydrolysis and ARSA activity within a range of 0.01 to 5.5 IU/L.

8. The analytical device of claim 1, wherein the analytical device is capable of providing results from actual serum and whole blood samples that are equivalent to those obtained from conventional standard methods.

9. The analytical device of claim 1, wherein the analytical device is disposable after a single use.

10. The analytical device of claim 1, wherein the paper substrate is a filter paper.

11. The analytical device of claim 1, wherein the analytical device is fabricated using a roll-to-roll printing process suitable for mass production.

12. A method for monitoring blood ARSA levels using the analytical device of claim 1, comprising:
applying a sample containing ARSA to the ion-selective electrode;
measuring a potential difference between the reference electrode and the ion-selective electrode; and
correlating the potential difference to an ARSA activity level in the sample.

13. The method of claim 12, wherein the sample is blood serum or whole blood.

14. The method of claim 12, wherein the potential difference is measured without use of mediators or pretreatment of the sample.

15. A method for fabricating a paper-based analytical device for potentiometric assay of ARSA, comprising:
patterning a paper substrate with wax to define zones for a reference electrode, an ion-selective electrode, and a sample application area;
printing electrodes on the paper substrate using an ink comprising carbon nanotubes and r-GO;
applying a polymeric membrane to the ion-selective electrode; and
integrating a solid-contact potentiometric sensor for ARSA with the electrodes.

16. The method of claim 15, wherein the polymeric membrane includes a molecularly-imprinted polymer specific for 4-NCS.

17. The method of claim 15, wherein the electrodes are printed using a stencil created by a laser cutter.
